(19) United States
(12) Patent Application Publication
(10) Pub. No.: US 2026/0256630 A1
(43) Pub. Date: Sep. 3, 2026

(54) PULL-UP DIAPER

(71) Applicant: RKW SE, Mannheim (DE)

(72) Inventors: Christoph WILLING, Vreden (DE); Robert KOPLIN, Dittenheim (DE); Reinhard EPPING, Gronau (DE); Ayhan SODO, Gronau (DE)

(73) Assignee: RKW SE, Mannheim (DE)

(21) Appl. No.: 19/163,594

(22) PCT Filed: Feb. 23, 2024
(86) PCT No.: PCT/EP2024/054629

§ 371 Date: Sep. 9, 2025

(30) Foreign Application Priority Data
Mar. 21, 2023 (DE) 10 2023 106 980.2

(51) Int. Cl. A61F 13/49

(52) U.S. Cl. CPC A61F 13/49012; A61F 13/4902

(57) ABSTRACT

A pull-up diaper (1) with a closed waist area (2), in which an elastic arrangement with at least one layer of nonwoven material (3, 4) is combined to form areas as a reserve (5). Bonding regions (6) are arranged between the elastic arrangement and the layer of nonwoven material (3, 4), wherein the elastic arrangement is designed as a flat structure in the form of an elastic film (7). The bonding regions (6) are designed as ultrasonic weld structures.

### CROSS-REFERENCE TO RELATED APPLICATIONS

This application is a 371 national phase of PCT/EP2024/054629, filed Feb. 23, 2024, which claims priority from German Patent Application No. 10 2023 106 980.2, filed Mar. 21, 2023, both of which are incorporated herein by reference as if fully set forth.

### TECHNICAL FIELD

The invention relates to a pull-up diaper with a closed waist area, in which an elastic arrangement is combined with at least one layer of nonwoven material to form areas as a reserve, with bonding regions arranged between the elastic arrangement and the layer of nonwoven material.

In pull-up diapers, an elastic, stretchable arrangement is used along a closed waist area to ensure a good fit and tightness. To increase wearing comfort, these diaper elements contain an elastic arrangement which ensures adaptation to the body shape by means of the retractive force of the elastic component. In conventional pull-up diapers according to the state of the art, the elastic arrangement comprises a plurality of elastic threads embedded between textile-like cover materials, such as nonwovens, to provide a pleasant feel.

### BACKGROUND

Such nonwoven fabrics generally have limited stretchability. In pull-up diapers, a nonwoven layer can therefore be partially bonded to the elastic arrangement in a corrugated form. The wave valleys are bonded to the elastic arrangement, while the wave peaks protrude away from the elastic arrangement and enclose a cavity toward the elastic layer.

EP 2 954 880 B1 describes a pull-up diaper with a closed waist area in which elastic threads are incorporated between layers of nonwoven material.

EP 0 556 749 B1 discloses a design for a pant-shaped diaper with a closed waist area, which is manufactured using an elastic film complex. The diaper comprises a main body formed from a liquid-impermeable layer and shaped like underpants. A belt-shaped element consisting of an elastic film complex is attached to the waist area of this main body. This elastic film complex is bonded to nonwoven fabrics by adhesive bonding.

The diapers and pull-up diapers described have a construction that allows them to be elastic and stretchy. However, for a high level of comfort and a snug fit to the adult body, waist areas or diaper bands that are not limited to uniform elasticity are desirable. This is particularly difficult to achieve with uniformly incorporated elastic threads.

### SUMMARY

The object of the invention is to provide a pull-up diaper with a closed waist area that adapts specifically to the anatomical needs of the wearer. To this end, the waist area should be anatomically designed and adjustable. Furthermore, the waist area should have favorable stretching properties while ensuring high tear resistance. The waist area should be stretchy when subjected to force, but also build up sufficient resistance to give the consumer the feeling of a high-quality product. The pull-up diaper should be harmless to health and ecologically sustainable. Furthermore, the waist area should have a pleasant feel. In addition, the pull-up diaper should prevent liquid from leaking.

This object is achieved by a pull-up diaper, a method and a use all having one or more of the features disclosed herein. Preferred variants can be found in the [[sub]] claims, the description, the examples of embodiment and the drawings.

According to the invention, the bonding regions are designed as ultrasonic weld structures.

For example, the elastic arrangement is designed as a flat structure, preferably in the form of an elastic film. The film is bonded to at least one layer of nonwoven material in a form-fitting and/or material-locking manner by ultrasonic welding. Variable feed behavior during the manufacture of the laminate from elastic film and at least one corrugated layer of nonwoven material allows regions with different areas as a reserve to be created along the waist area and fixed with ultrasonic weld structures.

The bonding regions can be produced using different methods. In a preferred embodiment of the invention, an already solidified elastic film is rolled off a film roll and a layer of nonwoven with defined corrugations is fed in, whereby the film and the layer are bonded to each other by bonding regions and the bonding regions are produced by ultrasonic welding. In this process, the corrugated nonwoven material is subjected to external thermal stress.

To this end, the nonwoven layer is rolled into a corrugated shape and welded to the elastic layer in the corrugation valleys.

In ultrasonic welding, a generator converts line voltage into high-frequency alternating voltage, which is converted into mechanical vibrations. The vibrations are transmitted to the elastic film and the nonwoven layer via a sonotrode, causing the material to heat up and become plasticized. After the sound waves have been applied, a short cooling phase under pressure ensures a homogeneous weld joint between the two parts. Ultrasonic welding is characterized by fast process times and high precision, while at the same time eliminating the need for additives and solvents.

In an alternative variant of the invention, the bonding areas can also be formed using an adhesive. A hot melt adhesive based on styrene block copolymer would be suitable for this purpose. Alternatively, a rigid hot melt adhesive can be used for spot application.

In the bonding regions, there is a form-fitting bond between the elastic material of the film and the nonwoven material. The material of the elastic film surrounds the filaments of the nonwoven layer so that, after the elastic layer has solidified, a form-fitting bond is created inside the bonding regions.

Between the bonding regions are areas in which the corrugated nonwoven layer protrudes in a wave-like pattern from the elastic film with its peaks and encloses cavities. These areas serve as a reserve for the expansion of the elastic arrangement, whereby there is no bonding between the nonwoven layer and the elastic film in these areas.

The term “elastic” for the elastic film preferably refers to a material that can stretch to an elongated length of at least approximately 160% of its relaxed original length when a directed force is applied, without tearing or breaking and which, after the applied force is removed, returns to at least 55% of its elongation, preferably substantially to its original length, i.e., the returned length is less than 120%, preferably less than 110%, more preferably less than 105% of the relaxed original length.

In a variant of the invention, the elastic film has at least one layer of polypropylene and/or an elastic polyolefin.

For example, the elastic film may also have a layer of SBC (styrene block copolymer) and/or TPE (thermoplastic elastomers). In an advantageous variant, the middle layer or core layer has a layer of SBC and/or TPE. In a variant of the invention, the elastic film has a multi-layer structure and is preferably designed as a coextruded film.

Styrene block copolymers (SBC) are polymers composed of polybutadiene or polyisoprene and polystyrene blocks. They are transparent plastics with high impact strength.

Optionally, the elastic film has outer layers. In one variant of the invention, an outer layer consists of a polypropylene. For example, a core layer of the elastic film is embedded between two outer layers. The outer layers made of polypropylene are particularly suitable for ultrasonic welding with a layer of nonwoven material.

For example, the ratio of core layer to outer layer is more than 10:1, preferably more than 11:1, in particular more than 12:1 and/or less than 15:1, preferably less than 14:1, in particular less than 13:1. In this way, weldability is particularly favorably balanced with the elastic properties.

The thickness of the elastic film was measured in accordance with DIN 53370 and given as an average value. In a favorable variant of the invention, the elastic film has a thickness of more than 10 μm, preferably more than 15 μm, in particular more than 20 μm and/or less than 70 μm, preferably less than 60 μm, in particular less than 50 μm. This makes the elastic film particularly thin and material-saving, while at the same time having an attractive feel.

In one variant of the invention, the layer consists of nonwoven polypropylene. The terms “nonwoven” and “fleece” refer to polypropylene which can be produced from continuous filaments and/or discontinuous fibers without weaving or knitting by means of processes such as spunbonding, carding, or meltblowing. The nonwoven fabric may comprise one or more layers of nonwoven, each layer containing continuous filaments or discontinuous fibers.

In one option of the invention, the elastic arrangement has a further layer of nonwoven.

For example, the elastic film is arranged between the two layers of nonwoven. Due to the wavy design, at least one layer of nonwoven is significantly longer than the elastic film. A second layer of nonwoven improves the feel of the pull-up diaper for the wearer.

The second layer of nonwoven material can also consist of a water jet-bonded nonwoven fabric, a carded nonwoven fabric, or a spunbonded nonwoven fabric. The second layer of nonwoven material can also be corrugated or have a flat profile. This gives the waist area a kind of cushion effect, the feel of which can be enhanced by the corrugated profile of the nonwoven layer.

In a variant of the invention, the elastic arrangement comprises a nonwoven layer made of a carded nonwoven fabric. The carded nonwoven fabric used preferably consists of polypropylene fibers and/or mixtures of different fiber types, such as polypropylene/viscose, polypropylene/polyamide, polypropylene/polyester, etc. The carded nonwoven fabric may also consist of polypropylene and/or polyethylene copolymer. Preferably, the specific weight of the carded nonwoven fabric is 10 to 40 g/m², in particular between 15 and 25 g/m². The carded nonwoven fabric may be consolidated, for example, by means of a calender and/or by exposure to air and/or a water jet.

Optionally, the elastic arrangement comprises a layer of nonwoven made from a spunbond. Spunbonds can be produced in very low grammages. The spunbond used preferably consists of polypropylene fibers and is in particular thermally bonded. Alternatively and/or in addition, the nonwoven layer may also comprise meltblown nonwovens. This nonwoven layer ideally has a specific weight of more than 4 g/m² preferably more than 5 g/m² particular more than 6 g/m² and/or less than 50 g/m², preferably more than 40 g/m² in particular more than 30 g/m²

A zone is synonymous with the terms area, section, and segment.

For example, the waist area comprises a first zone and at least one further zone, which have different areas as a reserve to ensure zone-specific flexibility under load. This is ideally achieved by the nonwoven layer within the zone having different proportions of bonding regions depending on the area to ensure zone-specific flexibility under load.

The form-fitting bond between the elastic film and the nonwoven layer creates bonding areas with high strength. In zones with a high proportion of bonding regions, this results in lower flexibility, whereas in bonding regions with a low proportion of bonding regions areas, higher zonal flexibility can be achieved.

The zone-specific variation in the proportions of bonding regions creates an elastic arrangement that better adapts to the anatomical needs of diaper wearers. This achieves a high level of wearing comfort by creating a zone with low flexibility and high conformability at the back and at least one zone with high flexibility in the hip area.

In the zones, the area-specific proportions of bonding regions of the nonwoven layer can be achieved by various design measures. These measures can also be combined with each other.

In one variant, the distances between the bonding regions of the first zone differ from the distances between the bonding regions of the further zone. In the zone with the smaller distances, the nonwoven layer is fixed to the nonwoven fabric in a surface-specific manner by a larger number of bonding regions.

Optionally, the bonding regions have point-like structures.

For example, the structures of the bonding regions are produced by the interaction of a sonotrode and the protrusions of a roller. In a preferred variant of the invention, the protrusions of the roller are designed as pyramid heads and produce structures that correspond to the negative of a pyramid head.

In a further variant of the invention, the structures of the bonding regions are circular and/or oval and/or oblong and/or rectangular and/or square and/or trapezoidal.

A structure is an arrangement and/or organization of interconnected elements in a material object.

For example, the bonding regions have structures that are arranged in a horizontal direction.

The horizontal direction extends along the waist area of the pull-up diaper. The structures of the bonding regions run along the waist area and provide the elasticity and snug fit of the incontinence article at the transition to the abdomen and back of the diaper wearer.

Optionally, the distances between the bonding regions within the structure can be varied to achieve zones with different areas as a reserve. For this purpose, the distances between the ultrasonic weld spot are varied and/or the folding of the nonwoven layer is adjusted to form the areas as a reserve.

Optionally, the bonding regions have structures that are arranged in a vertical direction.

The vertical direction extends across the height of the waist area of the pull-up diaper. The structures of the bonding regions can be designed with variable distances between each other so that zones with different retractive forces and body zone-specific conformability can be achieved.

For example, the bonding regions have structures that are offset from one another. In one variant of the invention, the horizontal and/or vertical structures of the bonding regions are offset from one another in the form of ultrasonic weld spots or ultrasonic weld line elements.

In an offset arrangement of the bonding region structures, the structures can be arranged on straight lines and/or diagonals that have interruptions.

Optionally, the structures of the bonding regions can be designed in the form of wave, checkerboard, or herringbone patterns.

For example, the waist area comprises a first zone and at least one further zone, which have different areas as reserve to ensure a zone specific compliance under load. For this purpose, the first zone may, for example, comprise areas as a reserve in the form of nonwoven fabric laid in waves, which has bonding regions with the elastic film in the wave valleys.

In addition, the further zone may preferably have no and/or very few areas as a reserve in the form of a corrugated layer of nonwoven material. In this embodiment, at least one and/or two layers of nonwoven material are bonded flush with the elastic film by means of bonding structures. In the embodiment described, this further zone has a very high retractive force.

For example, the ratio of the areas as reserve to ensure zone-specific compliance of the first zone to the further zone can be more than 2:1, preferably more than 3:1, in particular more than 4:1 and/or less than 20:1, preferably less than 15:1, in particular less than 10:1.

Opacity is the opposite of transparency. It is a measure of light impermeability or opacity in percent. In particular, the opacity of a completely light-impermeable film is 100%, and a completely or perfectly transparent film has an opacity of 0%.

In one variant of the invention, the elastic film has a print, whereby the nonwoven layer has a degree of transparency according to DIN 53416 of more than 85%, preferably more than 90%, in particular more than 95%, for the visibility of the print. This allows the print to shine through the nonwoven layer and is protected by the nonwoven layer.

In addition, the print can be designed as a print motif. In the field of film, the term print motif refers to the thematic design element of a print. If necessary, manufacturer-specific print motifs can also be included in the print.

A commonly used method for printing the elastic arrangement is flexographic printing. This is a direct relief printing process, also known as a rotary web printing process. The flexible printing plates, which are made of photopolymer or rubber, are used in combination with low-viscosity printing inks. The raised areas of the printing form carry the image. The advantages lie in the cost-effectiveness achieved through the use of a large printing width and high printing speed, as well as the availability of inexpensive printing inks. The printing tools, photopolymer printing plates and/or laser-engraved elastomer sleeves are readily available. Large print runs can be produced economically with flexographic printing. Printing is preferably carried out using flexographic printing. In addition, all other processes, such as offset gravure printing, are also included within the scope of the invention.

In an alternative version of the invention, the nonwoven layer can also be printed.

Digital printing is suitable for increasing the variety of designs while maintaining the economic efficiency of small print runs. The term covers a group of printing processes in which the print image is transmitted to a printing machine as digital data. Laser and inkjet printing are the most common methods. The major advantage of digital printing is that no static, unchangeable printing form is required. Instead, a pixel address is generated dynamically for each individual printing process within the print format, so that each printed copy can have a different print image if required.

The inkjet printing inks to be used for printing are preferably printing inks with a low viscosity and therefore almost as thin as water. In principle, aqueous, UV-curable, and solvent-based ink systems are suitable. These printing inks are specially adapted to the respective print heads and are therefore individually adapted to the respective printing machine.

The print motif is preferably printed with a water-based and/or glyco-containing composition in which the suitable pigments are dispersed. The printing ink can also be solvent-based, in which case a slow-drying solvent is most suitable. In addition, the ink may contain binders and additives.

Optionally, the elastic film of the pull-up diaper has a print. This print runs along the waist area and is preferably located in areas with little or no reserve space.

Advantageously, the visibility of the print is achieved by the transparency of the nonwoven layer in the printed areas.

According to the invention, a pull-up diaper is manufactured in a process in which at least one corrugated layer of nonwoven material with variable peaks and valleys is formed, an elastic film is extruded, and an elastic arrangement is produced as a flat structure by forming bonding regions in the form of ultrasonic weld structures between at least one layer of nonwoven material and the elastic film.

For example, in the production of the pull-up diaper, at least one layer of nonwoven material is brought into a three-dimensional, corrugated shape before the bonding step by passing it over a special device. This device may be a roller which has peaks and thereby forms the corrugated profile of the nonwoven layer. To produce the bonding areas between the nonwoven layer and the elastic film, a fabric bond and/or a form fit is created by ultrasonic welding. For this purpose, the nonwoven layer is rolled into a corrugated shape and welded to the elastic film in the corrugation valleys to form a structure.

For example, the waist area of the pull-up diaper has advantageous gas permeability in the first zone. The gas permeability is achieved due to the bonding regions in combination with stretching of the waist area, for example when the pull-up diaper is put on. This creates small zones of high gas permeability around the bonding regions.

Optionally, the waist area of the pull-up diaper comprises a first zone with high gas permeability and a further zone with very low gas permeability.

In a favourable variant of the invention, the proportion of the first zone is more than 50%, preferably more than 60%, in particular more than 70% of the area of the waist area. For example, the proportion of the further zone is more than 20%, preferably more than 30% in particular more than 40% of the area of the waist area.

According to the invention, a pull-up diaper with a closed waist area is used, which is made of an elastic film and at least one layer of nonwoven fabric bonded by ultrasonic weld structures to create a close fit to the body.

The realization of the bonding regions by means of ultrasonic weld structures enables a zone-specific retractive force of the waist area. This results in a waistband of a pull-up diaper that achieves an unprecedented degree of snugness compared to previous solutions with elastic threads.

### BRIEF DESCRIPTION OF THE DRAWINGS

Further advantages and features of the invention are apparent from the description of an embodiment with reference to the drawings and from the drawings themselves. This shows:

FIG. 1 a perspective view of a pull-up diaper,

FIG. 2 a sectional view of the waist area with uniform areas as a reserve,

FIG. 3 another cross-sectional view of the waist area.

FIG. 4 a third variant of the waist area in the sectional view,

FIG. 5 a fourth variant of the waist area in the sectional view,

FIG. 6 a schematic representation of the structures of the bonding regions,

FIG. 7 shows another variation of the structures, and

FIG. 8 a third variant of the structures.

### DETAILED DESCRIPTION

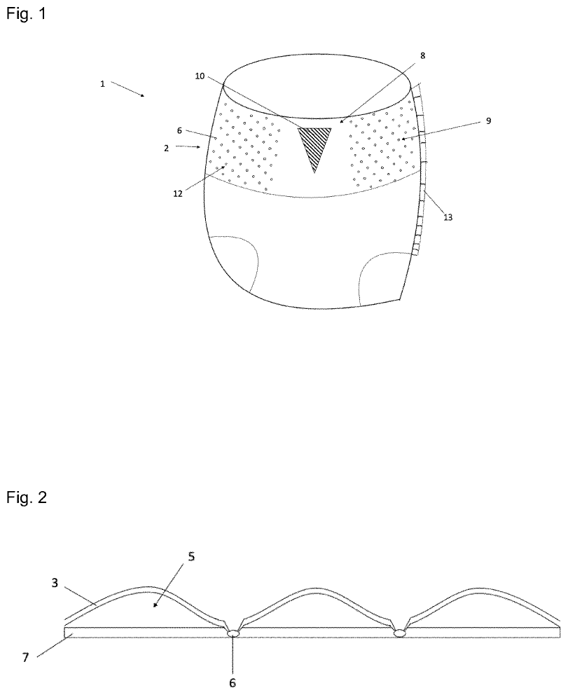

FIG. 1 shows a perspective view of a pull-up diaper 1 with a closed waist area 2. The pull-up diaper 1 is bonded on both sides to form a closed panty by means of the weld seam 13. The closed waist area 2 comprises a first zone 8 and at least one further zone 9.

Zone 8 has a print 10 with a manufacturer's logo and no areas as a reserve to ensure zone-specific flexibility under load, so that the print 10 is clearly visible through the nonwoven fabric 3.

Zone 9 has point-like structures 12 of the bonding regions 6, with the aid of which the elastic film 7 is bonded to at least one layer of nonwoven 3, 4 to form areas as a reserve 5. Zone 9 has a high degree of flexibility as well as a high retractive force, so that the waist area 2 fits snugly to the person wearing the pull-up diaper 1.

In the embodiment shown, the ratio of the areas as a reserve 5 to ensure zone-specific flexibility of zone 9 to zone 8 is 5:1.

In relation to the area of the waist area 2, the area of zone 9 is 75% and the area of zone 8 is 25%.

FIG. 2 shows a sectional view of the closed waist area 2 with uniform areas as a reserve 5. The elastic arrangement comprises an elastic film 7 and a layer of nonwoven fabric 3 made of a corrugated nonwoven fabric.

This is a thermally bonded spunbonded fabric, whereby a spunbonded fabric made of continuous filaments is used in the embodiment. The nonwoven layer 3 is corrugated before being joined to the elastic film 7. When the elastic film 7 is joined to the corrugated nonwoven layer 3, bonding regions 6 are created by ultrasonic welding, in which the nonwoven layer 3 and the elastic film 7 are joined by melting and binding solidification.

### FIRST EXAMPLE

In the embodiment, the elastic film 7 is shown as a multi-layer coextruded film with a core layer and two outer layers. The core layer consists of thermoplastic elastomers and the outer layer consists of polypropylene. The core layer has a thickness of 37 μm, while the thickness of the outer layer is 3 μm.

### SECOND EXAMPLE

The elastic film 7 is listed as a multi-layer coextruded film with a core layer and two outer layers. The core layer consists of thermoplastic elastomers and the outer layer consists of polypropylene. The core layer has a thickness of 25 μm, while the thickness of the outer layer is 2 μm.

The areas as a reserve 5 have no or only a very weak bond between the nonwoven layer 3 and the elastic film 7 and preferably enclose cavities.

In the embodiment, the web-to-groove ratio of the roller that forms the nonwoven layer 3 into a corrugated shape is 1:6, so that the distances between the bonding regions 6 in the embodiment shown are 3 mm.

Inside the bonding regions 6, there is a form-fitting and partially material-locking bond between the solidified elastic material of the elastic film 7 and the nonwoven layer 3. During this bonding process, the surface of the elastic film 7 is fused with the nonwoven fabric of the nonwoven layer 3.

The width of the point-like bonding regions 6 is between 0.1 and 1 mm, with a width of 0.5 mm being achieved in the embodiment shown.

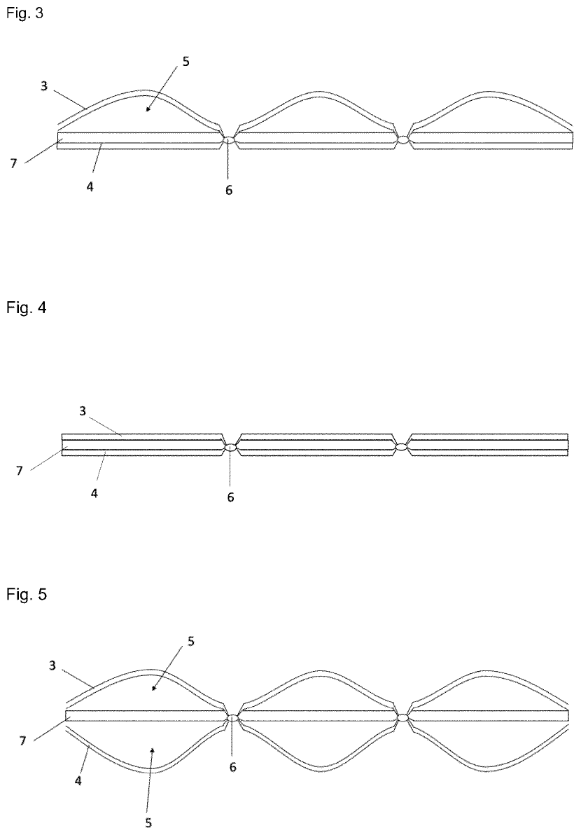

FIG. 3 shows a further sectional view of the waist area 2. The representation of the waist area 2 of zone 8 essentially corresponds to the description in FIG. 2. In addition, a further layer of nonwoven 4 is arranged on the side facing away from the areas as a reserve 5 and fixed by means of the bonding regions 6. The layer of nonwoven material 4 is smooth and not wavy on the elastic film 7. This allows print 10 applied to the elastic film 7 to be felt by observers through the layer of nonwoven material 4. At the same time, the layer of nonwoven material 4 protects the print 10.

FIG. 4 shows a third variant of the waist area 2 in a sectional view and essentially corresponds to the representation in FIG. 2. The layers made of nonwoven material 3 and 4 are smooth and not wavy and are bonded to the elastic film 7 by the bonding regions 6. This means that print 10 applied to the elastic film 7 or to the nonwoven layer 3 or 4 is visible from the outside.

FIG. 5 shows a third variant of the waist area 2 in a sectional view and corresponds essentially to the representation in FIG. 2. The waist area 2 has a zone 9 in which two layers of nonwoven material 3, 4 are arranged in a mirror image in a wave-like pattern around the elastic film 7 to form areas as a reserve 5 and are fixed by means of bonding regions 6. The bonding regions 6 are designed as ultrasonic weld spots which are produced by the contact of a roller with pyramid-shaped tips on a flat sonotrode. The bonding regions 6 are designed as ultrasonic weld spots, which are produced by the contact of a roller with pyramid-shaped tips on a flat sonotrode.

The nonwoven layers 3 and 4 are made of polypropylene and have a basis weight of 6 g/m². The nonwoven layers 3 and 4 have a transparency of 90%.

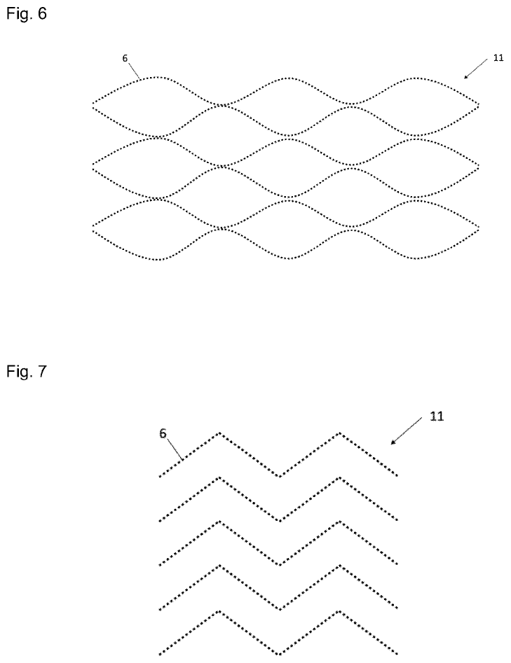

FIG. 6 shows a schematic representation of the structures 11 of the bonding regions 6 in the design as waves arranged mirror-inverted to each other. This achieves a high degree of elastic stretchability and thus favorable stretching properties of the closed waist area 2.

FIG. 7 shows a further variant of the structures 11 of the bonding regions 6 in the form of a herringbone pattern. This design variant achieves a favorable retractive force in the vertical direction of the waist area 2.

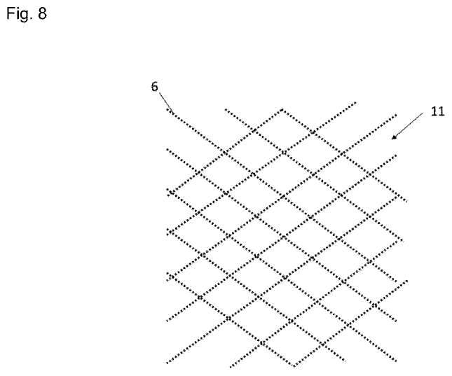

FIG. 8 shows a third variant of the structures 11 of the bonding regions 6 in the form of a checkerboard pattern. This design variant achieves a favorable retractive force in both the vertical and horizontal directions of the waist area 2.

1. A pull-up diaper (1), comprising:
a closed waist area (2); an elastic arrangement and at least one layer of nonwoven material (3, 4) combined to form areas as a reserve (5);
bonding regions (6) arranged between the elastic arrangement and the at least one layer of nonwoven material (3, 4);
the elastic arrangement comprises a flat structure formed as an elastic film (7); and
the bonding regions (6) comprise ultrasonic weld structures.

2. The pull-up diaper according to claim 1, wherein the elastic arrangement is arranged between two layers of the nonwoven material (3, 4).

3. The pull-up diaper according to claim 1, wherein the elastic film (7) comprises at least one layer formed of at least one of styrene block copolymers, polyolefin elastomers (POE), or thermoplastic elastomers (TPE).

4. The pull-up diaper according to claim 1, wherein the elastic film (7) has outer layers made of polypropylene.

5. The pull-up diaper according to claim 1, wherein the elastic film (7) has a thickness of more than 10 μm, and less than 70 μm.

6. The pull-up diaper according to claim 1, wherein the at least one layer of nonwoven material made of polypropylene.

7. The pull-up diaper according to claim 1, wherein the at least one layer of nonwoven material (3, 4) has a specific weight of more than 4 g/m² and less than 50 g/m².

8. The pull-up diaper according to claim 1, wherein the waist area (2) comprises a first zone (8) and at least one further zone (9) having different areas as the reserve (5) for ensuring zone-specific compliance under load.

9. The pull-up diaper according to claim 1, wherein the bonding regions (6) have point-shaped structures (12).

10. The pull-up diaper according to claim 1, wherein that the bonding regions (6) have structures (11) arranged in a horizontal direction.

11. The pull-up diaper according to claim 1, wherein the bonding regions (6) have structures (11) arranged in a vertical direction.

12. The pull-up diaper according to claim 1, wherein the bonding regions (6) have structures (11) that are arranged offset from one another.

13. The pull-up diaper according to claim 1, wherein the elastic film (7) has a print (10), the at least one layer of nonwoven material (3, 4) has a degree of transparency of more than 85%, visibility of the print (10).

14. A method for manufacturing the pull-up diaper (1) according to claim 1, comprising the following steps:
forming the at least one layer of nonwoven material as at least one corrugated layer of nonwoven material (3, 4) with variable peaks and valleys;
extruding an elastic film (7);
producing he elastic arrangement as a flat structure by forming the bonding regions (6) as the ultrasonic weld structures between the at least one layer of nonwoven material (3, 4) and the elastic film (7).

15. A method of using the pull-up diaper (1) of claim 1, comprising applying the with the closed waist area made of the elastic film (7) and the at least one layer of nonwoven material (3, 4), which are bonded by the ultrasonic weld structures, to a user for a close fit to a user's body.
